(19) United States
(12) Patent Application Publication
(10) Pub. No.: US 2022/0087891 A1
(43) Pub. Date: Mar. 24, 2022

(54) Systems And Methods For Multi Modal Personalized Topical Pain Relief

(71) Applicant: Modii Health Inc., Novato, CA (US)

(72) Inventors: Jonah Ross Goodman, San Francisco, CA (US); David Edward Goodman, Greenbrae, CA (US); Joseph Anthony Heanue, Oakland, CA (US)

(21) Appl. No.: 17/481,216

(22) Filed: Sep. 21, 2021

Related U.S. Application Data
(60) Provisional application No. 63/082,245, filed on Sep. 23, 2020.

(51) Int. Cl. A61H 7/00; A61N 1/36; A61N 1/04

(52) U.S. Cl. CPC A61H 7/00; A61N 1/36021; A61H 23/0245; A61N 1/0492; A61N 1/36031

(57) ABSTRACT

Taking an individualistic approach to the treatment of chronic pain may soon be the norm of modern-day medicine. The potential to control the type of medication, in addition to external stimulus including, but not limited to electrical, thermal, cold, or ultrasonic, presents itself in a new “toolbox.” The present invention relates to the management of chronic pain through non-opioid, multi modal compounded transdermal applications, whose properties are enhanced externally, by electrical, thermal, or mechanical energy. More specifically, the present invention encapsulates the customization of personal health, in analyzing, applying and contributing to the betterment of patient's well-being, adapting to patient's preference, biological requirements and regulating to various environments based on the individual's specific body part.

### CROSS-REFERENCE TO RELATED APPLICATIONS

The present application claims the priority benefit of U.S. Provisional Patent Application Ser. No. 63/082,245 filed on Sep. 23, 2020, and titled “A System and Method for Multi Modal Personalized Topical Pain Relief,” which is hereby incorporated by reference in its entirety.

### FIELD OF INVENTION

Exemplary embodiments pertain to acute and chronic personalized pain relief.

### SUMMARY OF EXEMPLARY EMBODIMENTS

Exemplary embodiments include a multi-modal personalized pain management system with a sensor subsystem within a single assembly, a skin stimulator subsystem within the single assembly, a drug delivery subsystem within the single assembly, a control subsystem within the single assembly, and a user interface subsystem. The single assembly may be configured to be attached or worn in a vicinity where pain relief is desired. The user interface subsystem includes software with instructions executed by a processor and configured to operate as part of the system or a separate computing device. The multi-modal personalized pain management system may include any of an oximeter, a pulse oximeter, a thermometer, an infrared radiation sensor, a colorimeter, a blood flow sensor, a pH sensor, a galvanic skin response sensor, an imbedded accelerometer, a biochemical sensor, an infrared radiation sensor, a colorimeter, a biochemical sensor, or a combination thereof.

The multi-modal personalized pain management system, according to exemplary embodiments, may further include the skin stimulator subsystem with an electrical stimulation portion, a thermal stimulation portion, a mechanical stimulation portion or any combination of an electrical portion, and a thermal portion, or a combination of an electrical portion and a mechanical portion, or a combination of a thermal portion and a mechanical portion, or a combination of an electrical portion, a thermal portion, and a mechanical portion. The electrical stimulation portion may induce a current flow to a tissue. The thermal stimulation portion may induce a temperature rise or fall in a tissue. The mechanical stimulation portion may apply a force to a tissue. Additionally, the control subsystem may be configured to cause a time-varying force.

Exemplary embodiments include the multi-modal personalized pain management system having any of a reciprocating electric motor, a rotating electric motor coupled to an eccentric weight, a piston driven by an external fluid such as air or water, a piezoelectric transducer, or any other means of applying a time-varying force. Additionally, the multi-modal personalized pain management system may include any of a drug dispensing mechanism, a drug dispensing control mechanism, a drug supply, a drug patch, a drug dispensing solid, a flat structure with a port and a channel, a trapped volume or any combination thereof. The drug delivery subsystem may be configured to dispense two or more types of drugs. The control subsystem may be configured to receive user inputs or sensed signals and to provide control over the skin stimulator subsystem or the drug delivery subsystem. The system may communicate over any of an Internet network, a radio frequency, near-field communication, optical communication, acoustic communication, or any other communication means capable of receiving sensed signals and transmitting control signals, or a combination thereof.

The multi-modal personalized pain management system, according to exemplary embodiments, also includes the user interface subsystem configured to provide information on functioning of the system and to provide information from a user on their preferences or perceptions as to the functioning of the system.

Also provided herein are exemplary methods for multi-modal personalized pain management, including receiving sensing input by a control subsystem in an assembly, receiving user input from a user interface by the control subsystem in the assembly, and generating a control signal by the control subsystem to control a skin stimulator subsystem in the assembly and a drug delivery subsystem in the assembly. Exemplary methods also include attaching the assembly in vicinity of where pain relief is desired and measuring by an infrared radiation sensor a combination of a temperature of skin and a temperature of a tissue beneath the skin. Various exemplary methods include measuring by a colorimeter any combination of skin color, tissue color beneath skin or color of a dye. A biochemical sensor may sense a substance exuded from skin, including any of water, sodium, potassium, oil, sebum, proteins including defensins, metabolic products including lactic acid, oxides of nitrogen, or a combination thereof.

### BRIEF DESCRIPTION OF THE DRAWINGS

In the description, for purposes of explanation and not limitation, specific details are set forth, such as particular embodiments, procedures, techniques, etc. to provide a thorough understanding of the present technology. However, it will be apparent to one skilled in the art that the present technology may be practiced in other embodiments that depart from these specific details.

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1. Shows an exemplary multi-modal pain management device (“MMPMD”).

FIG. 2. Shows an exemplary MMPMD with the user interface subsystem.

FIG. 3. Shows an exemplary sensor subsystem.

FIG. 4. Shows an exemplary infrared radiation sensor.

FIG. 5. Shows an exemplary colorimeter.

FIG. 6. Shows an exemplary biochemical sensor.

FIG. 7. Shows an exemplary skin stimulator subsystem.

FIG. 8. Shows an exemplary electrical stimulation portion.

FIG. 9. Shows an exemplary electrical stimulation portion.

FIG. 10. Shows an exemplary thermal stimulation portion.

FIG. 11. Shows an exemplary thermal stimulation device.

FIG. 12. Shows an exemplary mechanical stimulation portion.

FIG. 13. Shows an exemplary programmable motive device.

FIG. 14. Shows an exemplary programmable motive device.

FIG. 15. Shows an exemplary drug delivery subsystem.

FIG. 16. Shows an exemplary drug patch.

FIG. 17. Shows an exemplary drug patch.

FIG. 18. Shows an exemplary drug-dispensing solid.

FIG. 19. Shows an exemplary drug patch.

FIG. 20. Shows an exemplary drug delivery subsystem.

FIG. 21. Shows an exemplary dispensing control mechanism.

FIG. 22. Shows an exemplary drug delivery subsystem.

FIG. 23. Shows an exemplary situation where two or more drugs may be dispensed into the skin.

FIG. 24. Shows an exemplary control subsystem.

FIG. 25. Shows an exemplary control system.

FIG. 26. Shows an exemplary communication means.

FIG. 27. Shows an exemplary control subsystem.

FIG. 28. Shows an exemplary control subsystem.

FIG. 29. Shows an exemplary user interface subsystem.

FIG. 30. Shows an exemplary user interface subsystem.

### REFERENCE NUMERALS

1. Sensor Sub-System
2. Skin Stimulator Sub-System
3. Drug Delivery Sub-System
4. Control Sub-System
5. Contained Single System
6. Patient Pain Vicinity
7. User Interface
8. Pulse Oximeter
9. Colorimeter
10. Thermometer
11. Blood Flow Sensor
12. Galvanic Skin Response Sensor
13. Biochemical Sensor
14. Infrared Radiation Sensor
15. Medical Patch
16. Biochemical Imbedded Sheet
17. Electrical Stimulator
18. Mechanical Stimulator
19. Peltier Cooler (Thermal Stimulator)
20. Peltier Cooler (Cooling Stimulator)
21. Magnetic Coil: Electrical Stimulator
22. Mechanical (Vibrational Stimulus)
23. Mechanical (Time-Varying Force Stimulus)
24. Desired Medical Patch Placement
25. Medical Patch Adhesive
26. Diffusing Gel Matrix
27. Mobile Medical Patch
28. Capillary Flow Substance
29. Capillary Flow Substrate
30. Drug Channel
31. Drug Port
32. Combined Drug Port
33. Reduced Volume Channel
34. Aerosol/Vapor Channel
35. Contained Aerosol (Volume Dispensing Mechanism)
36. User Input
37. User Output
38. Algorithmic Control Signals
39. Separate Assembly
40. Radio Frequency Communication
41. Near Field Communication
42. Optical Communication
43. Acoustic Communication
44. User Input Controls
45. External Data Processing Capabilities
46. External Data Storage/Analysis (Cloud Based)
47. Data Results
48. Drug Delivery External Controller
49. Skin Stimulator External Controller
50. User Information

### DETAILED DESCRIPTION

While the present technology is susceptible of embodiment in many different forms, there is shown in the drawings and will herein be described in detail several specific embodiments with the understanding that the present disclosure is to be considered as an exemplification of the principles of the present technology and is not intended to limit the technology to the embodiments illustrated.

Chronic Pain has and continues to be a major challenge for the medical community. As a result, The US chronic Pain Relief Market is worth 21.97 billion and is expected to grow to $32.02 billion revenue by 2025, with a CAGR of 6.2%. Not only does chronic pain put a strain on the patient, but it also puts tremendous pressure on the physician. To figure out a way to get a patient's pain under control, in the least costly, the most targeted, and with the least addictive properties is a major challenge. However, most to all patients who suffer from chronic pain are treated with a multitude of medications, some of which are highly addictive and others that mask the entire body's pain receptors. These medications only work for a small percentage of patients, where the method of pain management does not target the point of pain, nor does it resolve the pain; as a result, roughly 20-30 percent of patients who were prescribed opioids misuse them, with 8-12 percent developing an addiction. There needs to be a more targeted way of controlling chronic pain.

Topical Analgesic-Based Pain Management:

Chronic pain is caused by a multitude of elements, such as an initial injury, back sprains, pulled muscles, etc. Therefore, the resulting pain is usually located in one specific part of the body, e.g. neck, back, etc. So, it's not inconceivable to try and treat pain where the pain originated, rather than masking pain over the whole body. There has been an increasing shift in the medical community on developing new and innovative ways of drug administration, that are customized to the individual patient. The use of Transdermal and Topical Compounded Custom Medication is one of the innovative avenues that primary care physicians take to treat chronic pain. For instance, Ketamine has shown tremendous promise to treat neuropathic pain as well as chronic pain of all types. Lidocaine and Gabapentin have shown great promise in treating general inflammation as well as neuropathic pain. And Baclofen a (NSAID) reduces substances in the body that cause pain and inflammation. Additionally, with companies such as Icy Hot, Bengay, Tiger Balm and others creating their own custom formulations of topicals to treat chronic pain, there are now many options for patients to customize their pain management regime. Since primary care physicians can now combine these medications to treat individualized ailments, there has been an increase in prescribing compounded medications. And with a 47% success rate and close to 0% chance of addiction, it is evident why primary care physicians are moving towards a more individualistic way of treating chronic pain.

Electrical Stimulus-Based Pain Management:

Another innovative way of treating chronic pain is through the use of a TENS (transcutaneous electrical nerve stimulation) devices, which is one of the most commonly used forms of electroanalgesia. These devices use low voltage, electric current to relieve pain and to target specific areas of pain. These devices can be used on any point on the body where there is pain and will send electric stimulus to the point of pain to contract and relieve the muscle. Other devices of this nature have been used such as heating pads, IcyHot's Smart Relief unit, and cooling units to target and treat chronic pain. The TENS device is also programmable, meaning you can code the system to have different modes depending on where its located. For instance, the back has thicker skin and have deeper muscles. Therefore, you could program a specific back algorithm to a TENS unit to target the muscles in the back. Same applies to the neck, hands, shoulders, etc. The theory is if you stimulate nerves, either through electric, thermal, or mechanical stimulus, you close a “gate” mechanism that can help eliminate the feeling of pain. While there is some discrepancy with the effectiveness of TENS units, patients generally have a 70-80 percent success rate in treating chronic pain after a few months or longer.

Cannabis-Based Pain Management:

Cannabis has been used medically for many purposes that span from treating epilepsy to stimulating a cancer patient's appetite. And only recently have people started applying the inflammation reducing properties of cannabis for treating chronic pain. However, since cannabis is not regulated by the FDA, there are large discrepancies over valid and invalid concentrations of CBD. But since pain is a purely subjective feeling, and over 100 million U.S. adults are plagued with chronic pain, this prompts patients to visit their doctors more and to test different modes of pain reduction. This is evident as CBD's starting revenue in 2018 was $350 million and product sales have grown in the United States to $590 million. It is predicted that the CBD market will grow to $117 billion dollars in 2025 with at a rate of 132%. As of 2018, hemp derived CBD is legalized in all 50 states and is already having a large impact on the way that people treat their pain. CBD may offer many options to patients who have had no success with their current medication regime. According to Eur J Pain, author of “Transdermal cannabidiol reduces inflammation and pain-related behaviors in a rat model of arthritis,” concluded that topical applications of CBD have been shown in some cases to inhibit neuropathic and inflammatory pain, which are extremely hard to treat. Of course, more research needs to go into to determine the efficacy of CBD, but there is no question that it is the next movement of supplemental medication.

Conclusion: A New Multi-Modal Pain Management System.

Since no medical profession contains the holy grail to treating chronic pain, different modalities will work in some cases and others will not. Therefore, having a device that combines different subsystems of treating chronic pain and incorporating them into one singular system would pose a great compliment to treating chronic pain.

### BRIEF DESCRIPTIONS OF DEVICE

Modii is a multi-modal pain management system that combines the therapeutic effects of transdermal medication and varying forms of external stimulation that include but are not limited to, electrical, thermal, mechanical and ultrasonic. The system uses machine learning to adjust skin stimulation based on qualitative measures (specific drug, user comfort, skin sensitivity, body placement). The system contains various imbedded sensors including but not limited to oximeters, colorimeters, pulse oximeters, optical sensors, hydration sensors, GRS sensors, accelerometers and PH sensors, and temperature sensors to monitor, analyze and promote skin health. Using sensors and circuitry to monitor changes (capacitive touch or resistive touch) in a particular state, the system can accommodate one or more drug releasing patches that can be placed in any custom arrangement. The properties of the patches can be interrogated to determine drug type, concentration, location, or depletion. The system could be controlled by a smart phone application which is used to operate all aspects of the system's stimulators, controllers, sensors and monitors. Using machine learning, the application will collect and analyze the patient's biometric data and eventually will make regimen recommendations based on positive experiences.

### DETAILED DESCRIPTION OF THE DEVICE

The skin thermal stimulator is a non-pharmaceutical, complementary pain management feature that can emit thermal heat with a maximum temperature of 43° C. Additionally, the thermal stimulator can also drop to a maximum temperature of −2° C. Depending on the injury the patient will want to use either cold or thermal stimulation to either increase or decrease the skins permeability rate, control the depletion of certain drugs, or for comfort. Thermal emission is used to dilate the blood vessels of inflamed muscles to promote blood flow and help sore and tightened muscles relax. Cold emission is used to reduce inflammation by decreasing blood flow.

The skin mechanical/ultrasound stimulator is a non-pharmaceutical, complementary pain management feature that can emit high-frequency sound or vibrational waves. For the ultrasound stimulator, 80 dBA is used for a cumulative exposure of 24 h over a 24 h period; an offset of 3 dBA is to be added to this value when halving the cumulative exposure time over a 24 h period (e.g. 83 dBA for 12 h over a 24 h period); 140 dB un-weighted BC (peak) sound pressure level for impulsive or impact acoustic energy (noise). For the vibrational stimulator, the max vibration is 2.5 m/s2 for a cumulative time of 8 h during a 24 h period. Both mechanical and ultrasonic vibration has shown tremendous effectiveness in relieving sore muscles, increasing muscle range of motion, increasing blood blow and improving muscle strength.

Adjustable Stimulation Controller

The device has an adjustable stimulation controller, operated by a smartphone application, which can be calibrated to the individual needs of the patient. The external stimulation controller can also be adjusted to either increase or decrease the skins permeability rate, in conjunction with the adjusting the speed at which the drug is delivered. For instance, if certain medications take longer to absorbed into the skin; the patient has the ability to increase the thermal stimulation to both aid in muscle relaxation and skin absorption. The device's stimulation controller is powered by machine learning, that is able to learn, adapt and adjust stimulation based on different qualitative measures (specific drug, user comfortability, skin sensitivity, body placement). For example, the back muscles are protected by thick skin and would need a more intense stimulation level for the patient to achieve a therapeutic effect. Therefore, through individual patient use and feedback, the devices AI will determine which stimulation/stimulation level achieves the most pain relief, per the individual. The stimulation controller also has a limit to how much stimulation can be applied to the skin. The stimulation will not go over FDA's ISO 60601 limits for maximum allowable voltage, current, temperature, vibration, and cold.

Skin Sensors

The system will contain a multitude of Skin Sensors to monitor, analyze and promote continuous skin health. The system will contain a Pulse Oximeter to measure oxygen saturation levels. Oximeters can determine small changes in how oxygen is being carried throughout the extremities of the body. The systems contain an embedded oximeter to determine how well the heart is pumping oxygen throughout the body, especially in the area where the system is placed.

The system will contain an infrared temperature sensor to determine the base temperature of the patient's skin and make sure that there are no dramatic spikes that cause skin irritation or damage.

The system will contain a colorimeter, which is a light sensitive device, used to determine how much of the medication has been depleted. Each medical patch will contain a dye, that when interacts with an external stimulus, changes color as it's absorbed into the skin. The colorimeter will track the color change in the patch and report the percentage depleted overall. Additionally, the colorimeter will also determine how well blood is flowing through the body, by measuring the transmittance and absorption of light that is passed through the affected area.

The system will contain optical sensors for the detection and placement of medical patches. The device will support a custom mosaicking the medical patches, almost like a patch quilt, so the patient can easily assemble a custom array of medical patches. These optical sensors will be placed along the bottom of the device and will be able to determine the type of medication, the dosage, the percentage depleted and the location. The system will contain hydration and a PH sensor for individuals wanting to monitor the hydration status of their skin, in real time. The sensor will monitor the patient hydration vitals, particularly in elderly patient, and will update them on whether or not they are in need of hydration. The device will use a PH sensor to determine that the skin has a balanced PH. The PH sensor will be able to measure the about of alkalinity and acidity on the skin. The system will contain an Accelerometer to determine where on the patient body, the system has been placed and the relative motion of the location. The system is able to be placed on any part of the body.

Skin Drug Monitor

By using hydrogel adhesives, the system can attach to any desired body part. Similarly, the medical patches can be placed on any part on the adhesive side of the system. Each patch will contain a custom barcode that can be read and recorded through a smartphone camera. Once the barcode is read and the app records the patch information, the patient has complete freedom to place the patch anywhere on the device.

The system's gel matrix contains a resistive touch circuit and will be able to record the exact location of the patch. Patient customizability will arise from the ease of attaching and detaching different patches onto the device, in a series of unique configurations. Once a patient attaches a patch to the device, the device will sense the patch and record the location, medication, percentage depleted and dosage of patch.

During each session, the system will record the duration, the type of stimulus, the intensity of the stimulus, the medications used, the amount of medication depleted and location on the body. The system has the ability to modify its gel matrix to control the rate of medication depletion. For instance, a doctor might recommend using a heat stimulus to increase the skins permeability and therefore, absorbing more medication. On the contrary, a patient may use a cold stimulus to slow the rate of absorption to extend the patch's duration. This will come from the devices drug dose controller, that can create a custom treatment plan based on the type of medication and the custom required rate of depletion.

The system will be able to measure drug depletion via an optical sensor. The colorimeter will determine how much medication is left in the patch and will recommend to the patient to switch it out. Each patch will contain a dye, that when exposed to an external stimulus (heat, cold, electric) will change color. The optical sensor will analyze the color change and determine the percentage of medication left.

### DETAILED DESCRIPTIONS OF THE FIGURES

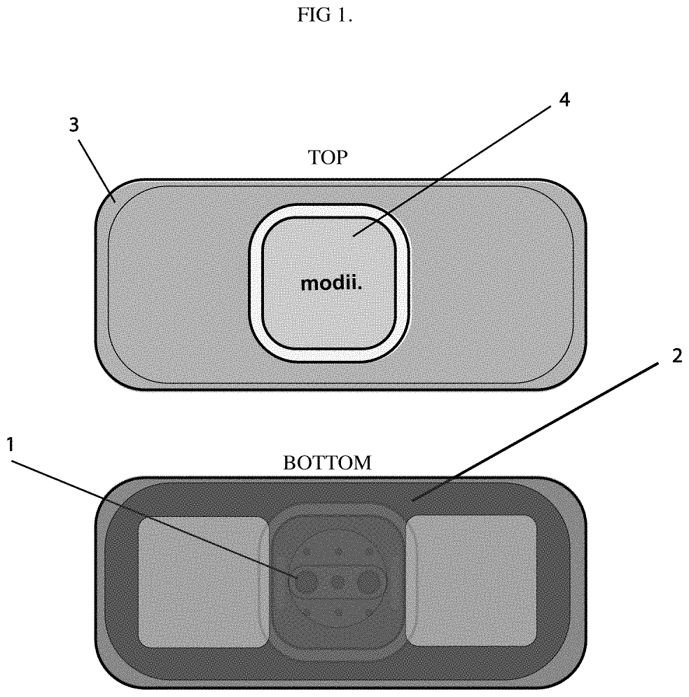

FIG. 1. The multi-modal pain management device (MMPMD) comprises typically four subsystems, which are a sensor subsystem, a skin stimulator subsystem, a drug delivery subsystem, a control subsystem and a user interface subsystem. Typically, a control subsystem receives sensing input from a sensor subsystem as well as user input from a user interface subsystem, and then generates appropriate control signals that control a skin stimulator subsystem and a drug delivery subsystem.

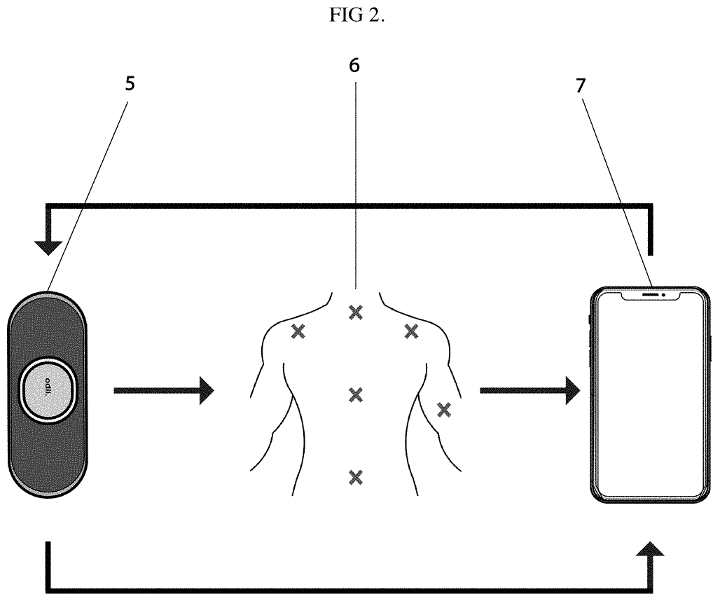

FIG. 2. Typically, the subsystems of the MMPMD with the possible exception of the user interface subsystem is contained within a single assembly which assembly is attached to or worn by the patient in the vicinity of where pain relief is desired. The user interface subsystem may be embodied as software running on the MMPMD or on an alternative device such as a smart phone.

Sensor Subsystem

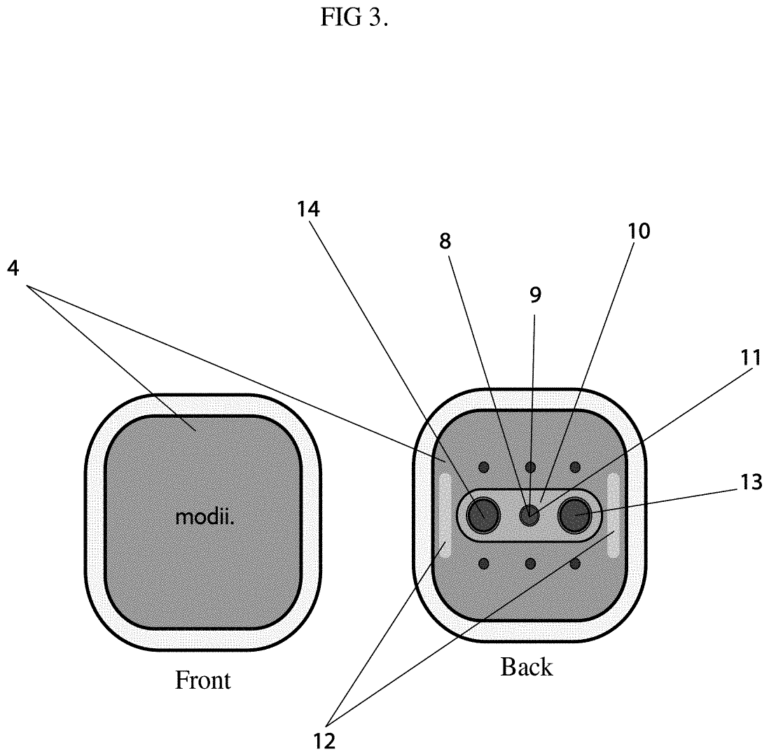

FIG. 3. A sensor subsystem may comprise a combination of sensors from the set of an oximeter or alternatively a pulse oximeter; a thermometer; an infrared radiation sensor; a colorimeter; a blood flow sensor; a pH sensor; a galvanic skin response sensor; an imbedded accelerometer; a biochemical sensor. Sensors provide sensing signals to the control subsystem.

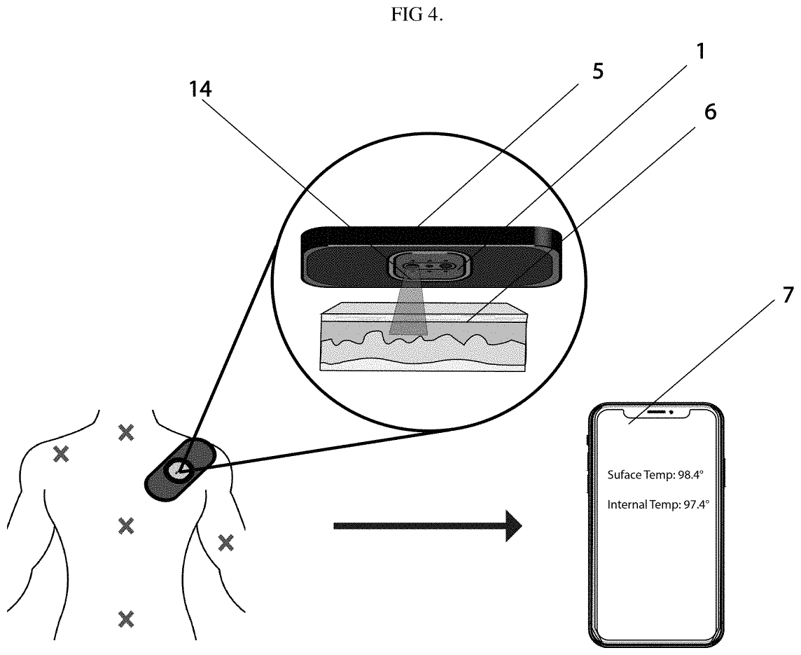

FIG. 4. In alternative embodiments, an infrared radiation sensor may measure a combination of the temperature of the skin and the temperature of tissues beneath the skin. The results of infrared radiation sensing are one or more sensing signals.

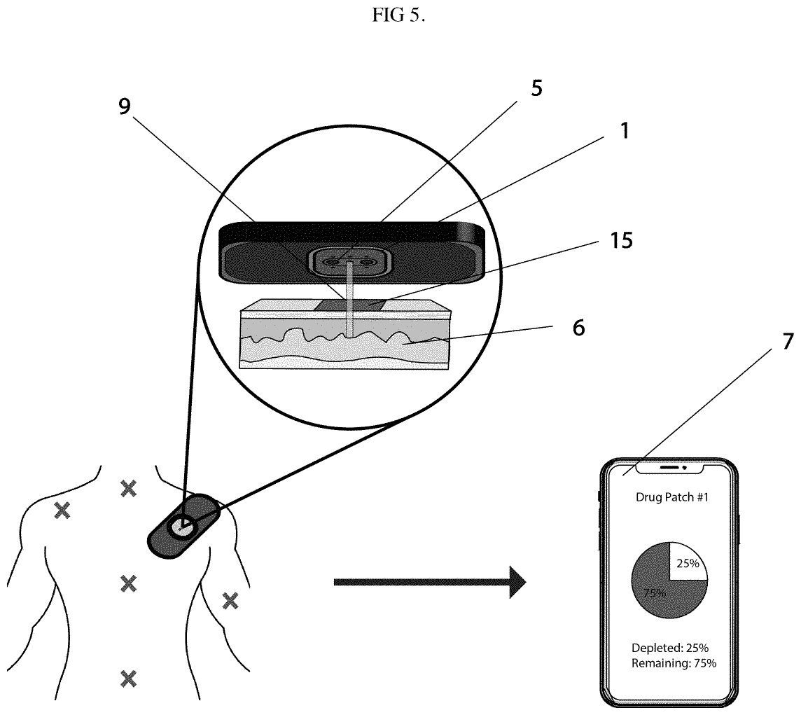

FIG. 5. In alternative embodiments a colorimeter may make measurements of any combination of the color of the skin, the color of some tissues beneath the skin, or the color of a dye. In additional embodiments a dye may be dispensed into the tissue in relationship to the amount of drug dispensed into the tissue. The results of colorimetry are one or more sensing signals.

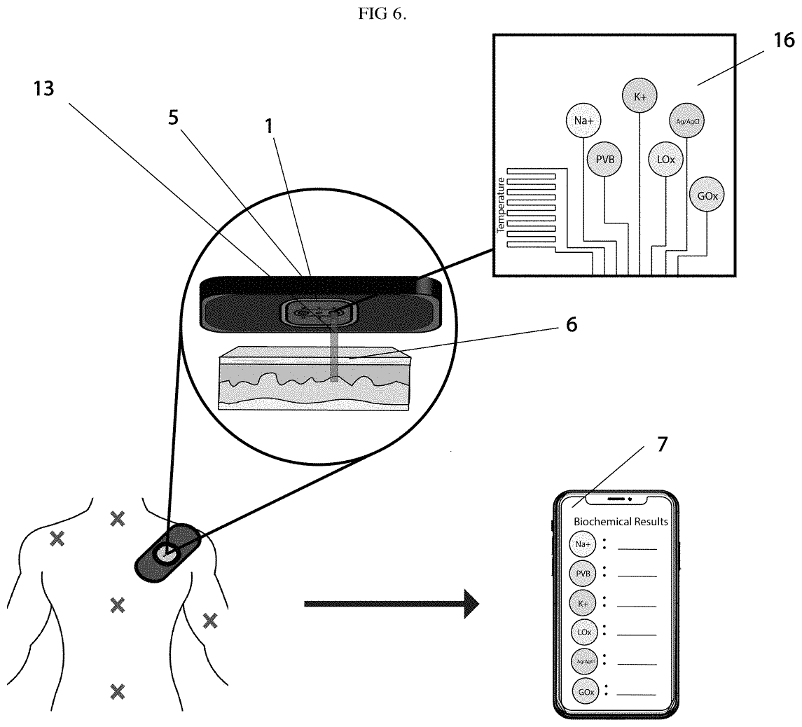

FIG. 6. In some embodiments, a biochemical sensor senses substance that are exuded from the skin. Some sensed substances are water, sodium, potassium, oil, sebum, proteins including defensins, metabolic products including lactic acid, oxides of nitrogen. The results of sensing substances are one or more sensing signals.

Skin Stimulator Subsystem

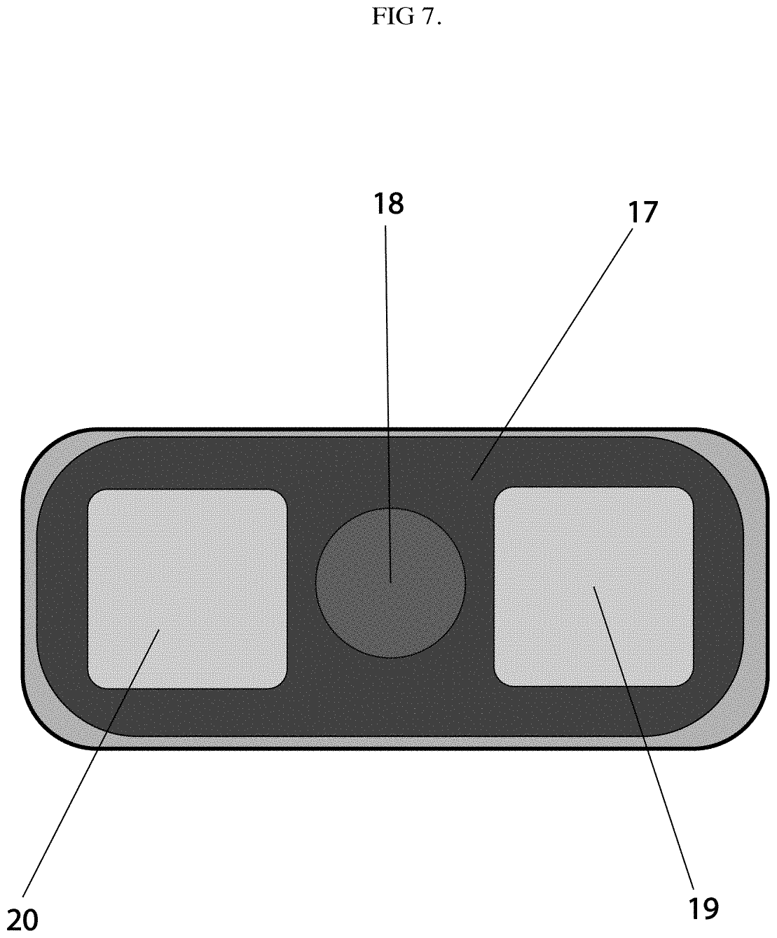

FIG. 7. A skin stimulator subsystem may comprise an electrical stimulation portion, a thermal stimulation portion, a mechanical stimulation portion or any combination of an electrical portion, and a thermal portion, or a combination of an electrical portion and a mechanical portion, or a combination of a thermal portion and a mechanical portion, or a combination of an electrical portion, a thermal portion, and a mechanical portion.

Electrical Stimulation Portion

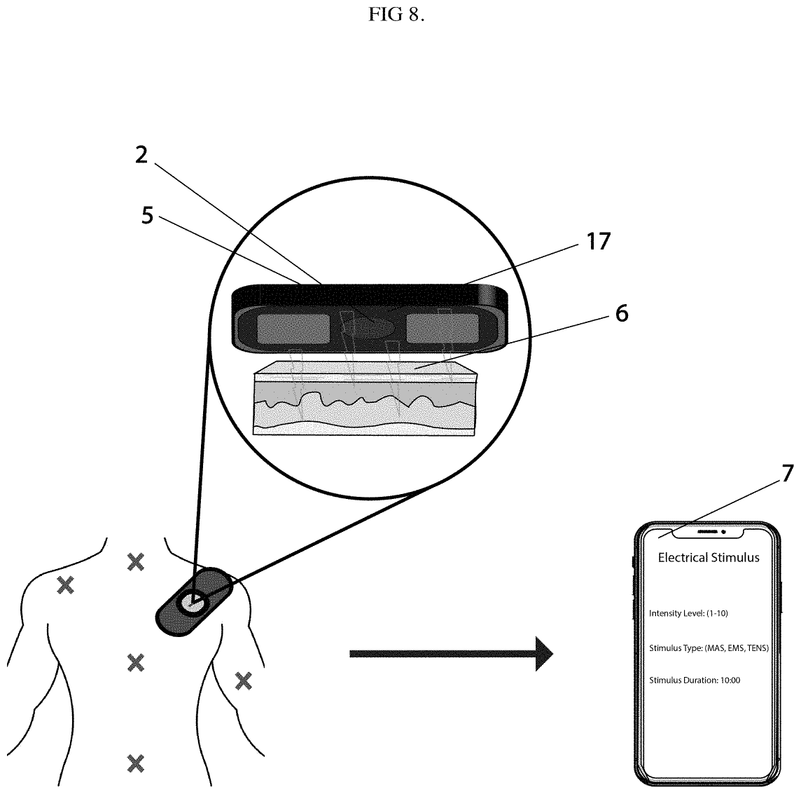

FIG. 8. An electrical stimulation portion comprises a means to induce current flow in tissues to be stimulated. Tissues to be stimulated may include skin, muscle, adipose tissue, nerve tissue, or any other tissues proximate to the skin surface. In one alternative embodiment, a time-varying electrical voltage is applied by programmable electrical circuits to at least two electrode patches that are in electrical contact with the skin lying over the tissues to be stimulated. The programmable electrical circuits are programmed to cause the electrical voltage to have a desired time-variation. The electrode patches in contact with the skin cause electrical currents to flow in the tissues to be stimulated causing stimulation to those tissues. It is to be understood that through a combination of the physical shape of the electrode patches with the programming of the electronic circuits, a wide variety of physical and temporal distributions of electrical current may be caused to flow in the tissues to be stimulated, in turn providing a variety of stimulation patterns to be applied to the tissues. Such stimulation patterns can be chosen to cause stimulation that is desirable from the standpoint of managing pain. In various embodiments, the total electrical current induced by the electrode patches may range from 1 mA to 200 mA peak. In some alternative embodiments, to assure the safety of the patient wearing the MMPMD, the relative voltage differences that may be applied to the electrode patches are limited. Such limits may be 20 mV.

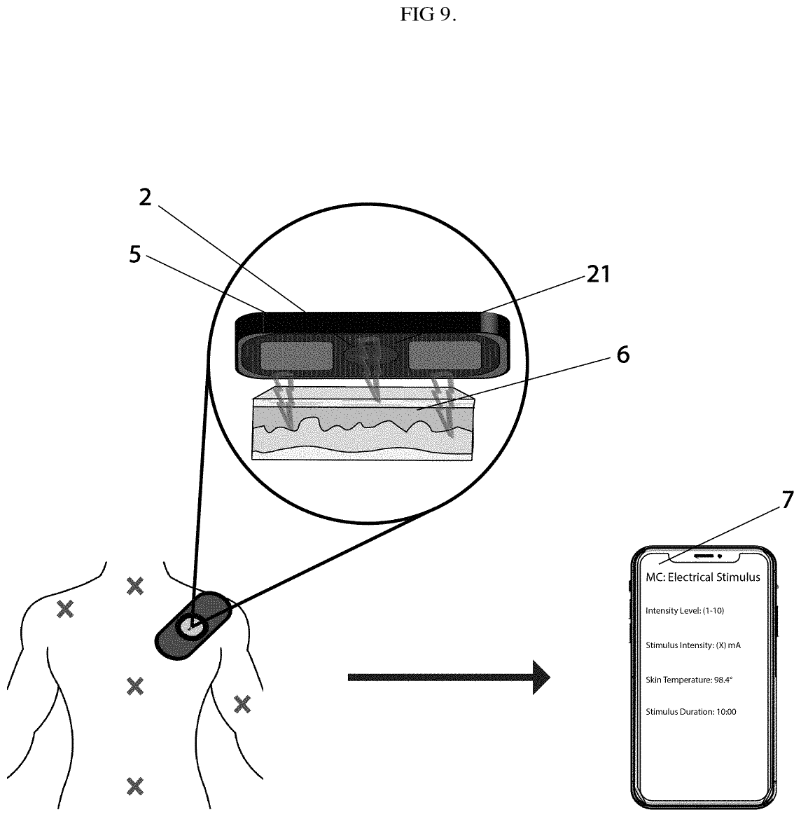

FIG. 9. In an alternative embodiment of the electrical stimulation portion, a magnetic coil may be placed adjacent to the skin overlying tissues to be stimulated. A time-varying electrical current is driven by programmable electronic circuits through the coil where the programmable electronic circuits are programmed to cause the electrical current to have a desired time-variation. The time-varying electrical current causes the magnetic coil to generate a corresponding time-varying magnetic field that partially penetrates the adjacent skin and the tissues to be stimulated. The time-varying magnetic field causes a corresponding time-varying electric field to be induced in the tissues to be stimulated. The induced time-varying electrical field causes to flow electrical current in the tissues to be stimulated causing stimulation to those tissues. It is to be understood that through a combination the physical shape of the magnetic coil and the programming of the electronic circuits, a wide variety of physical and temporal distributions of electrical current may be caused to flow in the tissues to be stimulated, in turn providing a variety of stimulation patterns to be applied to the tissues. Such stimulation patterns can be chosen to cause stimulation that is desirable from the standpoint of managing pain.

Thermal Stimulation Portion

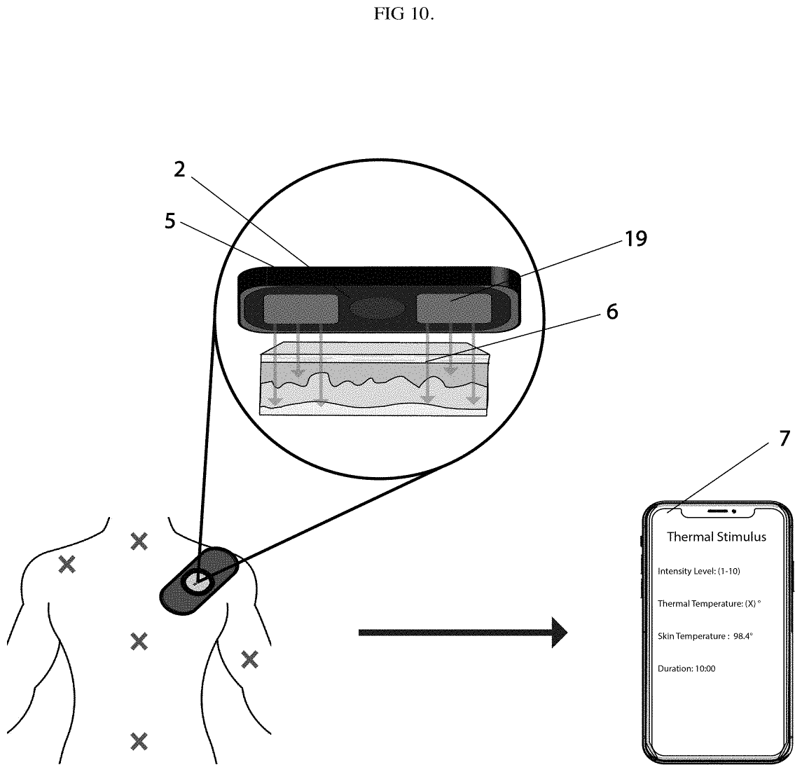

FIG. 10 A thermal stimulation portion comprises a means to induce temperature rise or fall in tissues to be stimulated. Tissues to be stimulated may include skin, muscle, adipose tissue, nerve tissue, or any other tissues proximate to the skin surface. In some alternative embodiments, the thermal stimulation portion may comprise a heating surface placed in contact with the skin. The heating surface is connected to a source of higher temperature such as an electrical heater, a heater supplied with hot gas, a heat pump, or a thermoelectric device arranged to cause the temperature of the surface of the heating surface to rise. The heating surface, the temperature of which is rising, causes the temperature of skin to rise, and by means of thermal conduction causes the temperature to rise of the tissues to be stimulated. The source of higher temperature may be controlled, for example, by the control subsystem to cause heating that is efficacious in managing pain. In additional embodiments, the source of higher temperature may be controlled such that the high-temperature limit tolerable by skin is not exceeded. In additional embodiments the size and shape of the heating surface can be chosen to produce thermal stimulation to a portion of the skin and hence to a desirable portion of the tissue to be stimulated.

Cold Stimulation Portion

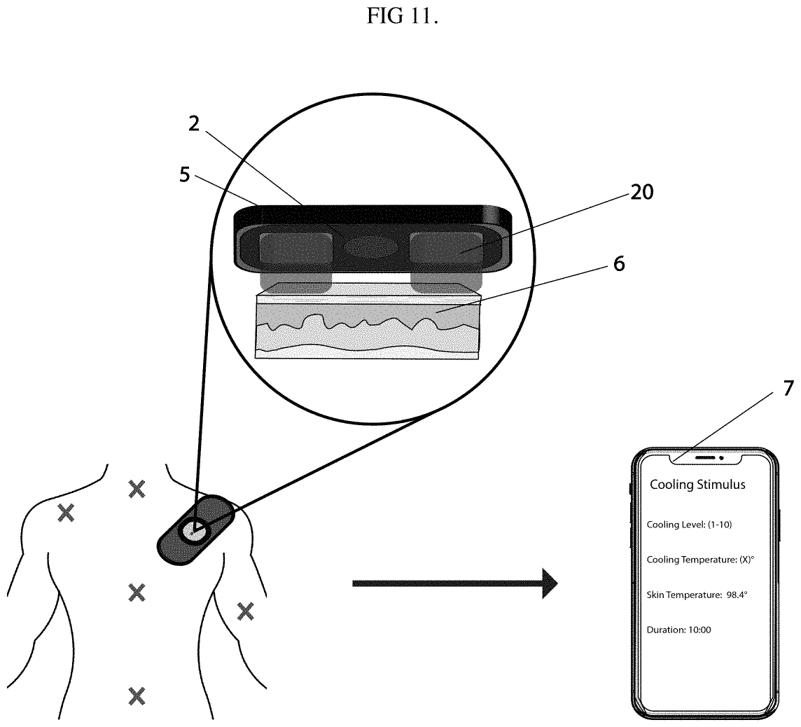

FIG. 11. In alternative embodiments, the thermal stimulation device may comprise a cooling surface placed in contact with the skin. The cooling surface is connected to a source of lower temperature such as a supply of cold gas, a heat pump, or a thermoelectric device, arranged to cause the temperature of the surface of the cooler to decrease. The cooling surface, the temperature of which is falling, causes the temperature of skin to decrease, and by means of thermal conduction causes the temperature to fall of the tissues to be stimulated. The source of lower temperature may be controlled, for example, by the control subsystem to cause cooling that is efficacious in managing pain. In additional embodiments, the source of lower temperature may be controlled such that the low-temperature limit tolerable by skin is not exceeded. In additional embodiments the size and shape of the cooling surface can be chosen to produce thermal stimulation to a portion of the skin and hence to a desirable portion of the tissue to be stimulated.

Mechanical Stimulation Portion

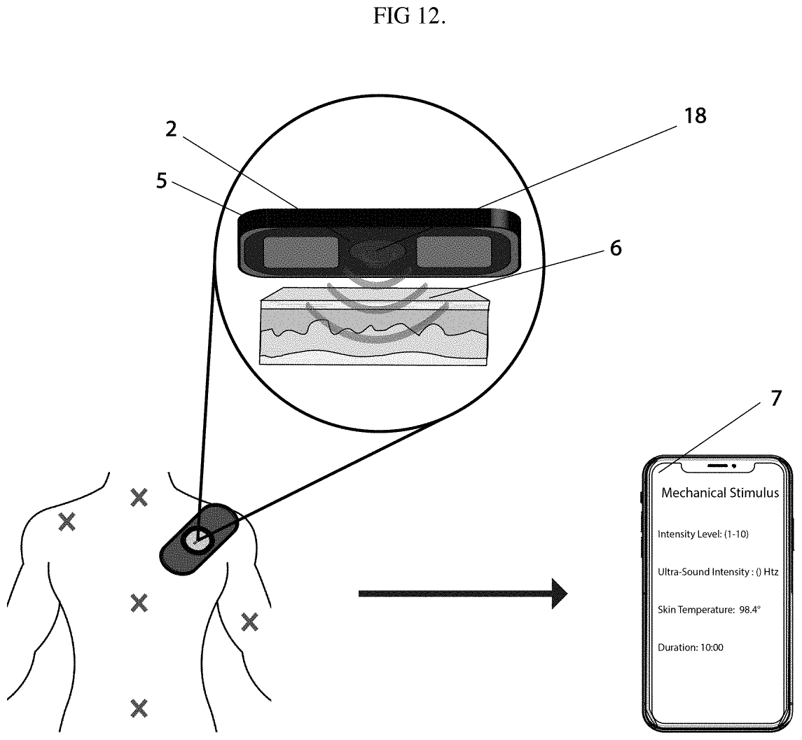

FIG. 12. A mechanical stimulation portion comprises a means to induce force to be applied to tissues to be stimulated. Tissues to be stimulated may include skin, muscle, adipose tissue, nerve tissue, or any other tissues proximate to the skin surface. In some embodiments, the mechanical stimulation portion comprises a surface placed adjacent to the skin together with a programmable motive device that applies a time-varying force to the surface. The surface transmits the force to the skin, which by virtue of the elastic properties of the tissue, causes the time-varying force to be transmitted to the tissues to be stimulated. In some additional embodiments the shape of the surface can be designed to provide mechanical stimulation to a portion of the skin and hence to a desirable portion of the tissue to be stimulated.

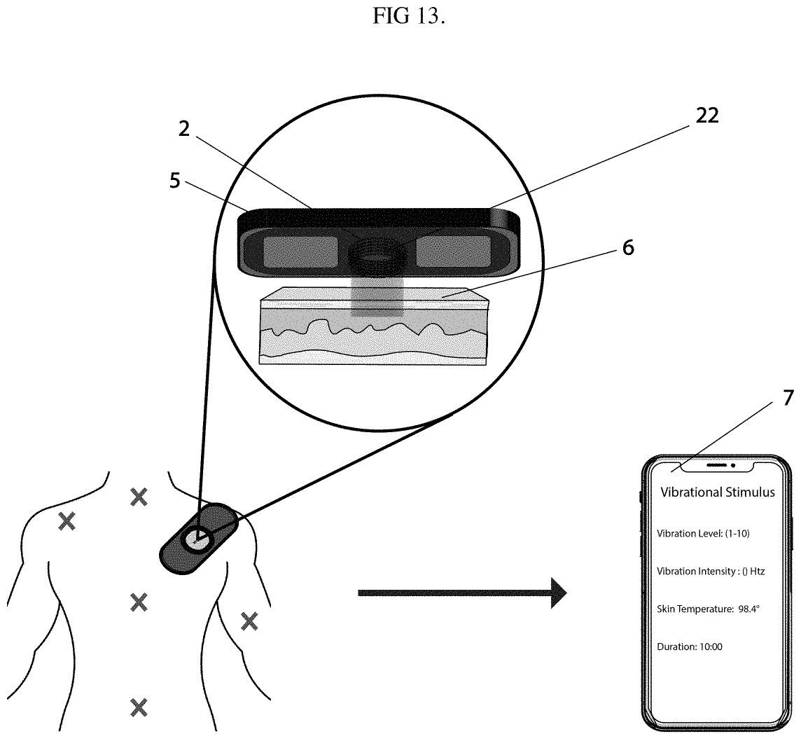

FIG. 13. In additional embodiments the programmable motive device may be controlled, for example, by the control subsystem to cause a time-varying force that is efficacious in managing pain. In further additional embodiments, the programmable motive device may be controlled to cause the time-varying force not to exceed limits tolerable by the skin or the tissue to be stimulated.

In alternative embodiments, the time-varying force oscillates such that it is periodic in time. Such an oscillating force the frequency of oscillation may be in the audio range from 20 Hz to 15 kHz, or in the sub-audio range from 0.1 Hz to 20 Hz, or in the ultrasonic range from 15 kHz to 1 MHz.

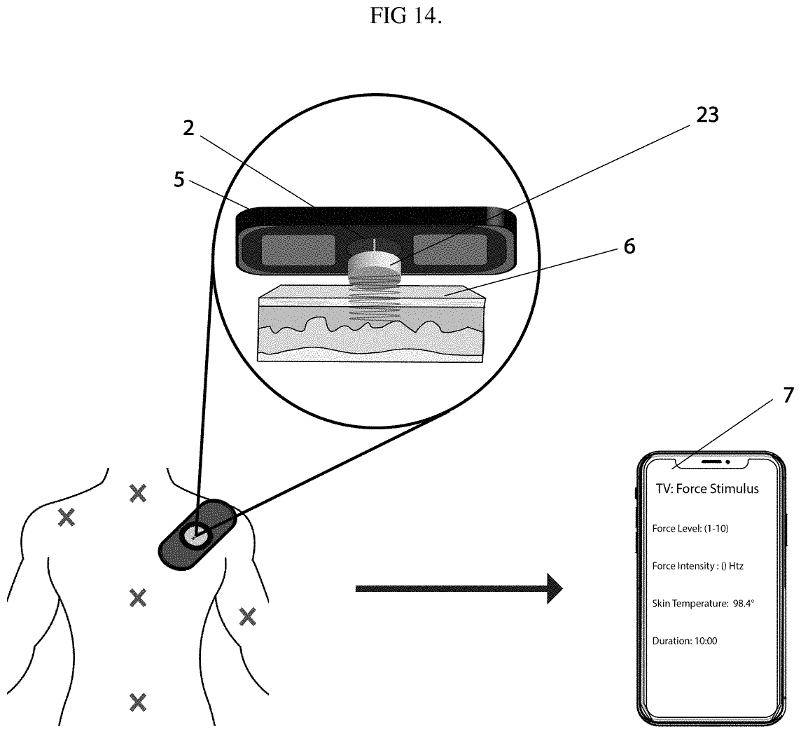

FIG. 14. In alternative embodiments the programmable motive device may be a reciprocating electric motor, a rotating electric motor coupled to an eccentric weight, a piston driven by an external fluid such as air or water, a piezoelectric transducer, or any other means of applying a time-varying force. In the case of a piston driven by an external fluid, the external fluid may be supplied by a pump, a speaker such as a loudspeaker, or any other means of supplying an external fluid. In alternative embodiments, aspects of the skin stimulator subsystem may be controlled by the system controller such that the skin stimulator subsystem does not interfere with accurate measurements made by the sensor subsystem.

Drug Delivery Subsystem

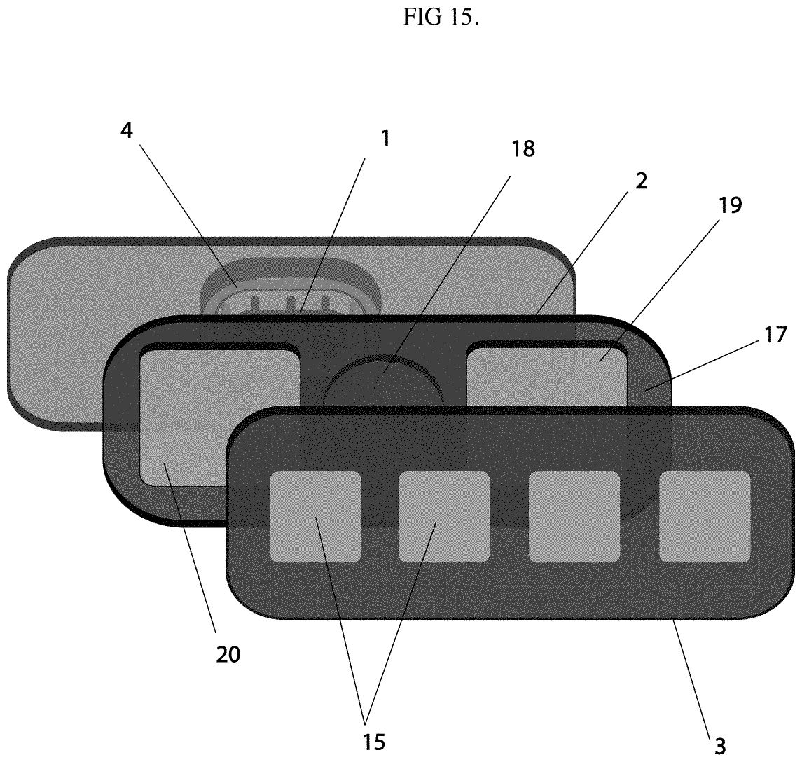

FIG. 15. A drug delivery subsystem may comprise one or more of a drug supplies, a drug dispensing mechanism, or a dispensing control mechanism.

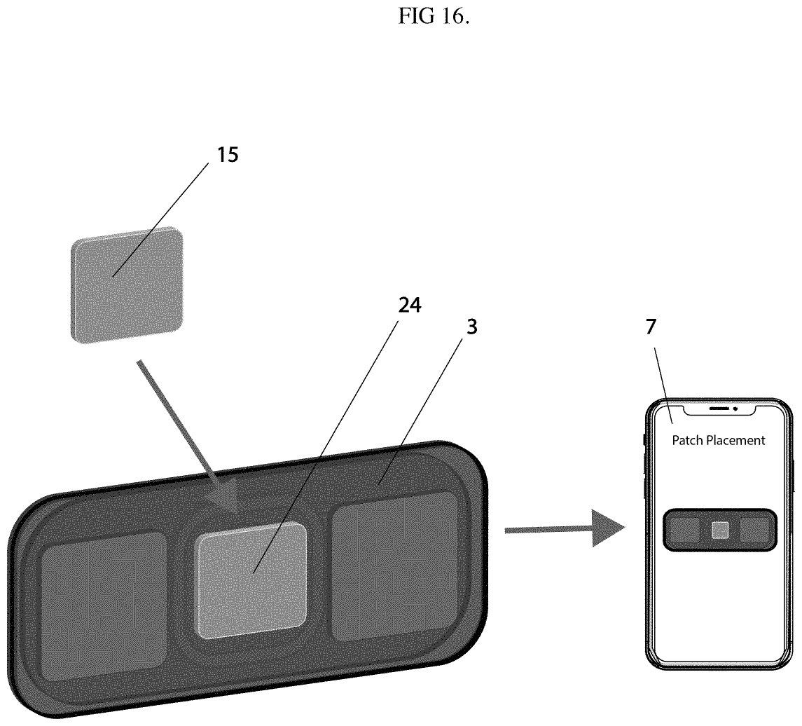

FIG. 16. In some embodiments, a drug patch comprises a drug supply, a drug dispensing mechanism and a dispensing control mechanism. A drug patch comprises at least a flexible substrate and drug-dispensing solid. In some embodiments, a drug patch is held such that the drug-dispensing solid is positioned at a desirable location and in contact with the skin.

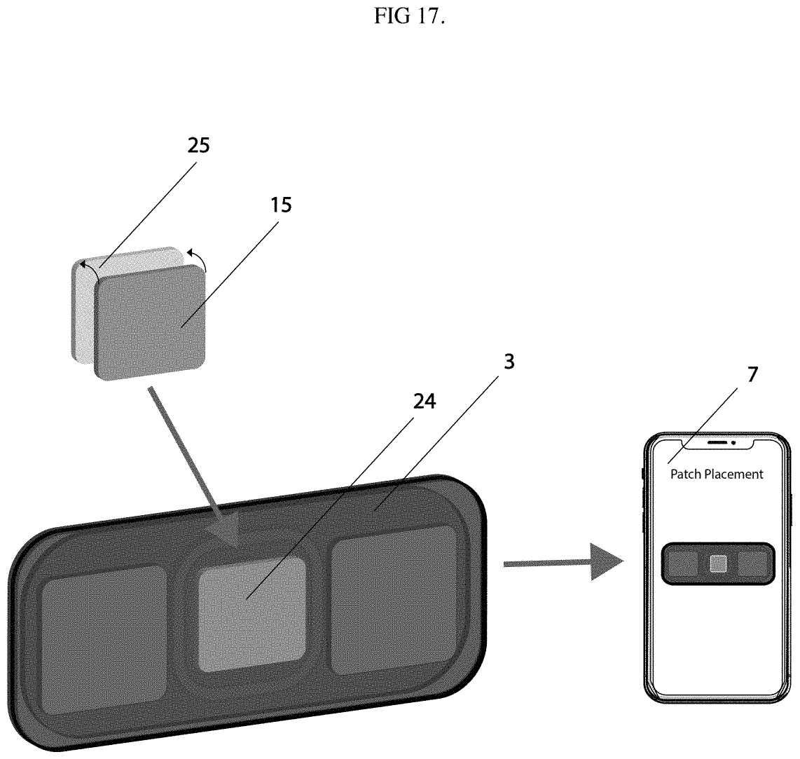

FIG. 17. In some embodiments, the drug patch comprises also an adhesive that holds the drug-dispensing solid in contact with the skin. In alternative embodiments, the drug patch is held by mechanical force. Mechanical force may be pressure or compression, or any other mechanical force that holds the drug patch in a desirable place.

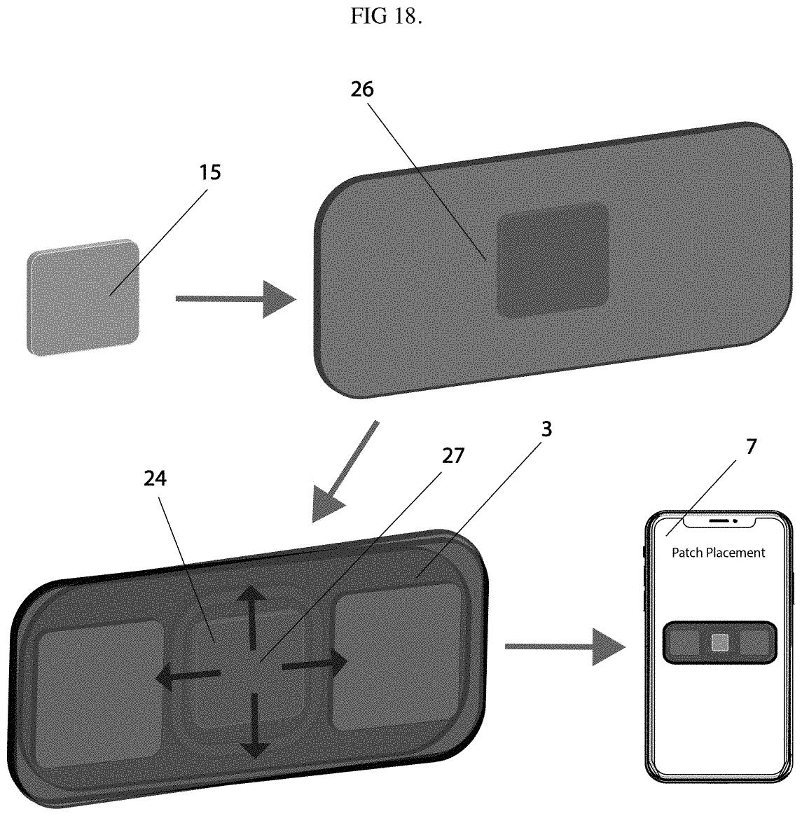

FIG. 18. In some embodiments, a drug-dispensing solid is a gel-like substance which is flexible, can hold drug thus providing a drug supply, and in which the drug is mobile. The drug being mobile allows the drug to diffuse through the gel-like substance to the interface between the drug patch and the skin, whereupon some of the drug may migrate from the patch to the skin thus providing a drug-dispensing mechanism. In some embodiments, a dispensing control mechanism comprises the rate at which drug may migrate from the patch to the skin.

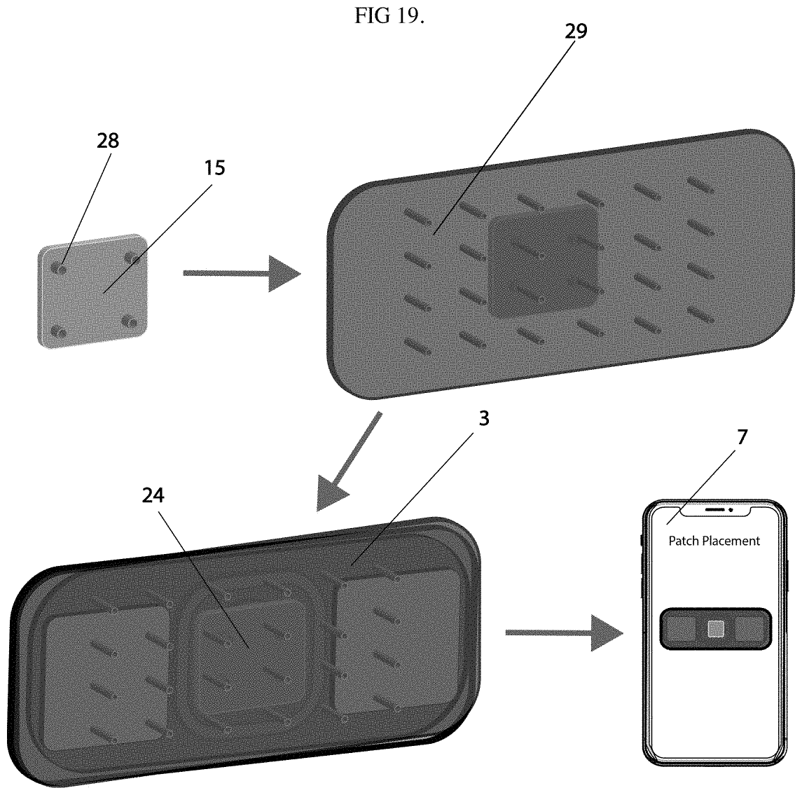

FIG. 19. In alternative embodiments, a drug patch comprises at least a substance providing capillary flow within the substance, which is a capillary flow substance. In further embodiments, drug is dispensed from a drug supply into a capillary flow substance, whereupon the drug is distributed throughout the capillary flow substance. In further embodiments, the capillary flow substance is held in contact with the skin, whereupon some of the drug may migrate from the patch to the skin thus providing a drug-dispensing mechanism. In some embodiments, a dispensing control mechanism comprises the rate at which drug may migrate from the patch to the skin. In alternative embodiments, a dispensing control mechanism comprises the rate at which drug is dispensed from a drug supply into the capillary flow substance.

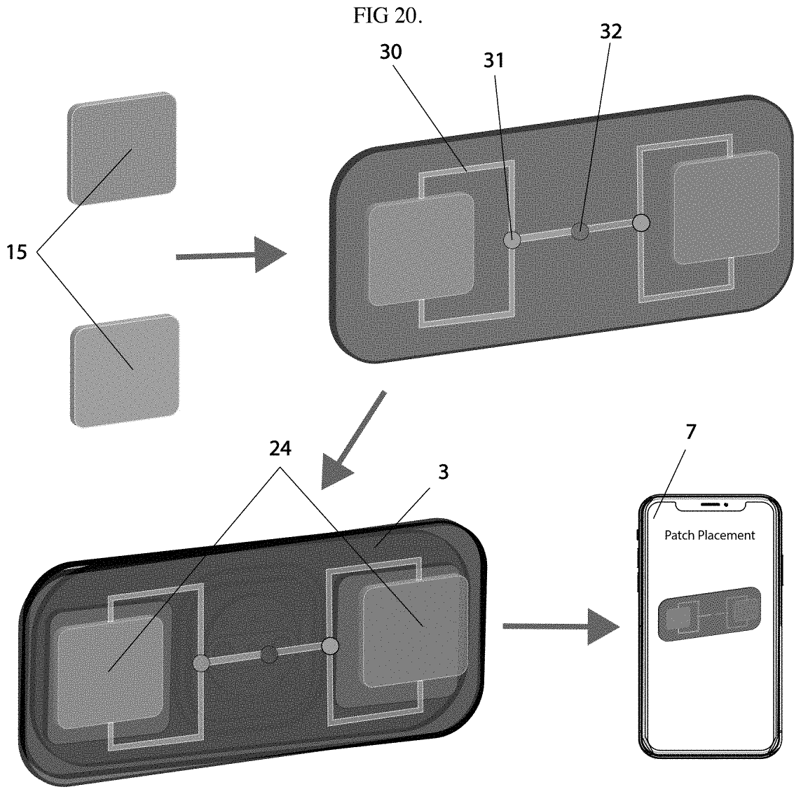

FIG. 20. In some embodiments, a drug delivery subsystem comprises at least a flat structure which further comprises one or more channels and one or more ports. Channels connect ports. Channels allow flow of drug from at least a first port, the input port, to at least a second port, the output port. One or more output ports may be positioned such that the opening they provide is directly adjacent to the skin. Drug that flows through at least one channel to at least one output is in contact with the skin and maybe taken up by the skin, providing a drug dispensing mechanism.

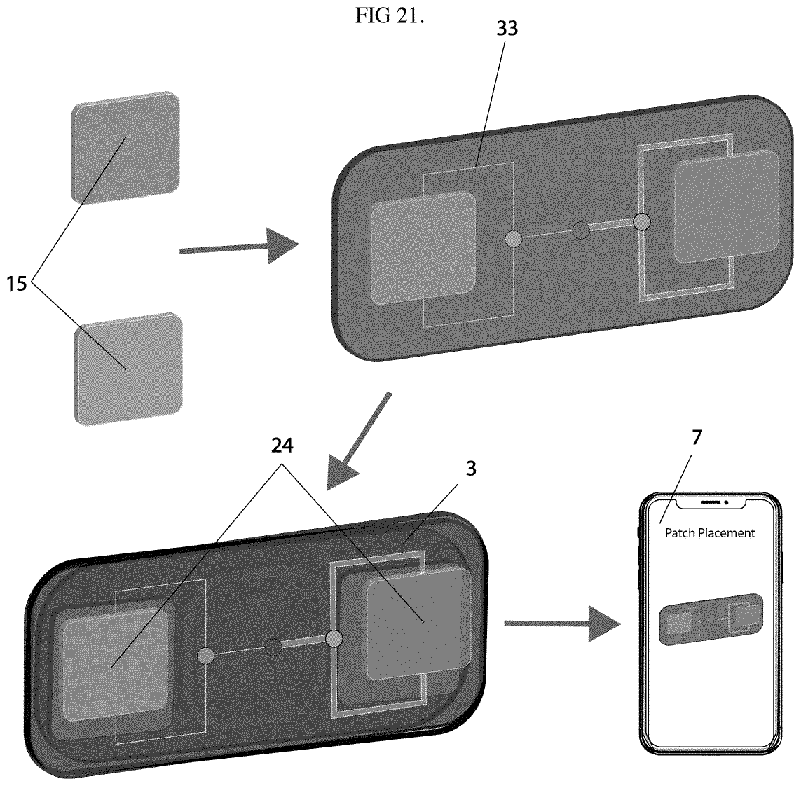

FIG. 21. In further embodiments, a dispensing control mechanism comprises the rate at which drug is introduced into at least one input port. In further embodiments at least one channel is constructed to have a very small enclosed volume, thus allowing minimal quantity of drug to remain in the channel un-dispensed.

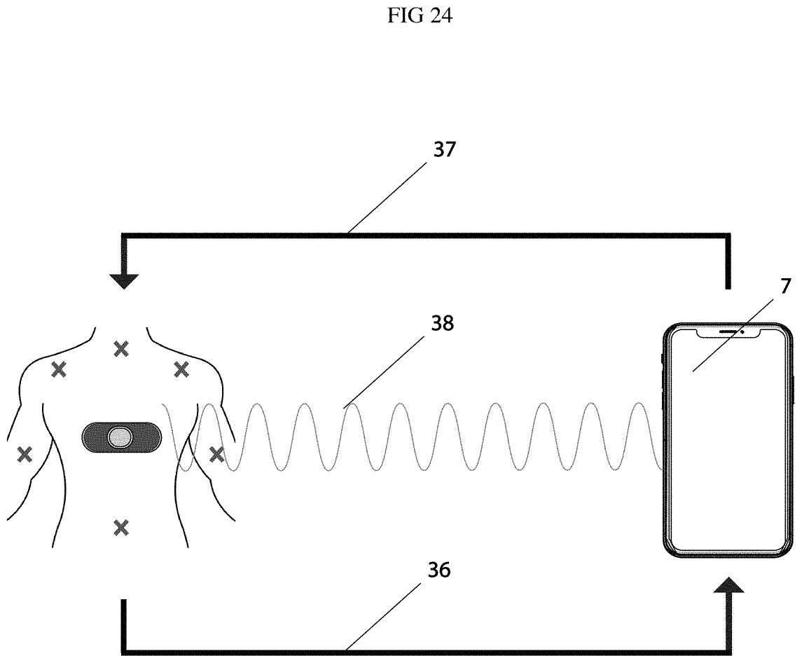

FIG. 22. In some embodiments, a drug delivery subsystem comprises at least a trapped volume, one boundary of which consists of the skin. A drug dispensing mechanism comprises the introduction of a vapor or aerosol containing the drug into the trapped volume. By diffusion the vapor or aerosol will distribute substantially throughout the volume. Further, the vapor or aerosol may condense or deposit on the skin, whereupon the drug may be absorbed by the skin. In further embodiments, a dispensing control mechanism comprises the rate at which vapor, or aerosol is introduced into the trapped volume.

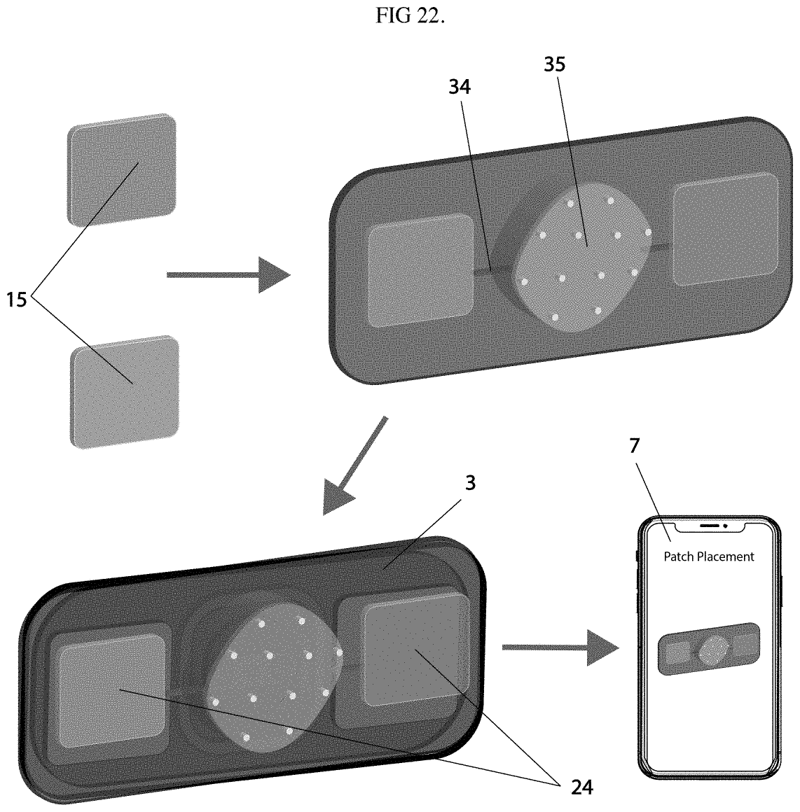

FIG. 23. In some embodiments, two or more drugs may be dispensed into the skin. Any of the means described above may be employed to dispense these two or more drugs. In further embodiments, each of the two or more drugs is controlled by corresponding dispensing control mechanisms.

Control Subsystem

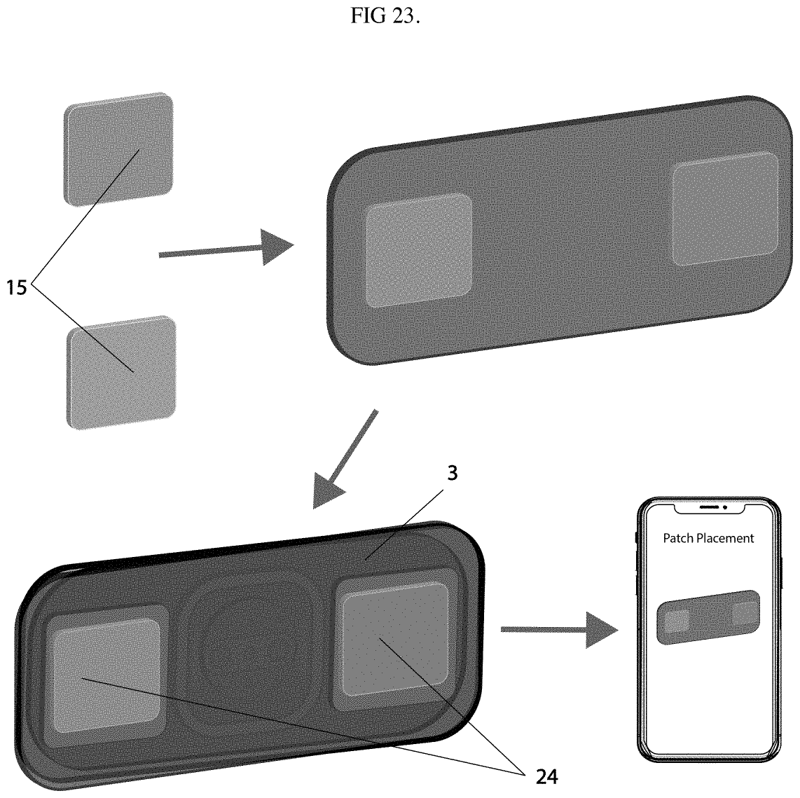

FIG. 24. A control subsystem may comprise any means by which to receive user inputs or sensed signals and to provide control over a skin stimulator subsystem or a drug delivery subsystem. In some embodiments, a control subsystem comprises a microprocessor or microcontroller or any other programmable electronic control system. In further embodiments, algorithms control a skin stimulator subsystem or a drug delivery subsystem in view of user inputs or sensed signals. In some embodiments, a control system comprises analog and digital electronics which receive user inputs or sensed signals and via analog circuitry generates control signals which control a skin stimulator subsystem or a drug delivery subsystem.

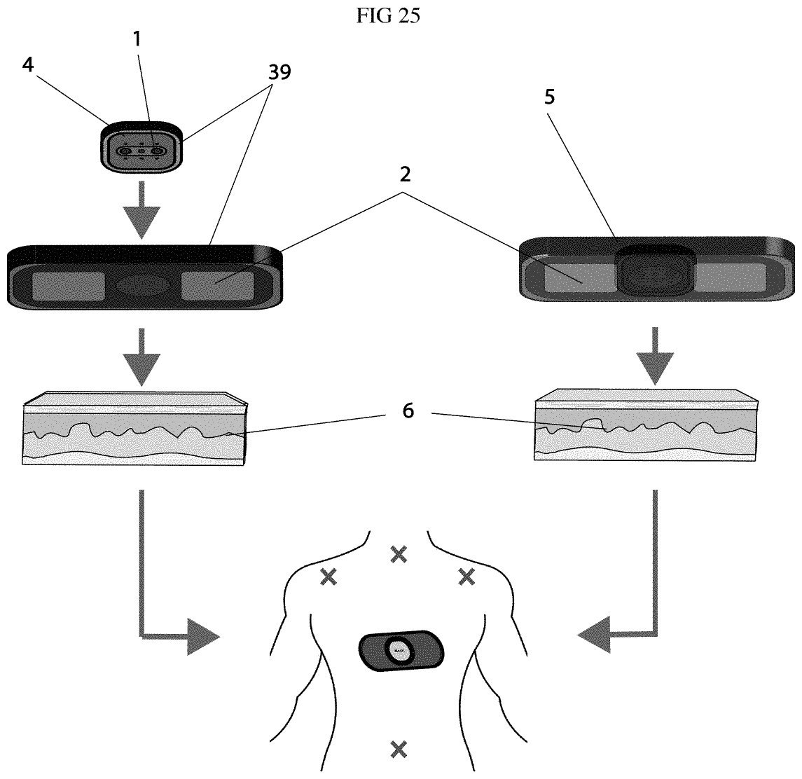

FIG. 25. In some embodiments, a control system is incorporated into the assembly that is applied to the patient's skin. In alternative embodiments, a control system resides in a container that is separate from the assembly that is applied to the patient's skin, providing a separate controller. In further embodiments a separate controller receives sensed signals and transmits control signals via communication means.

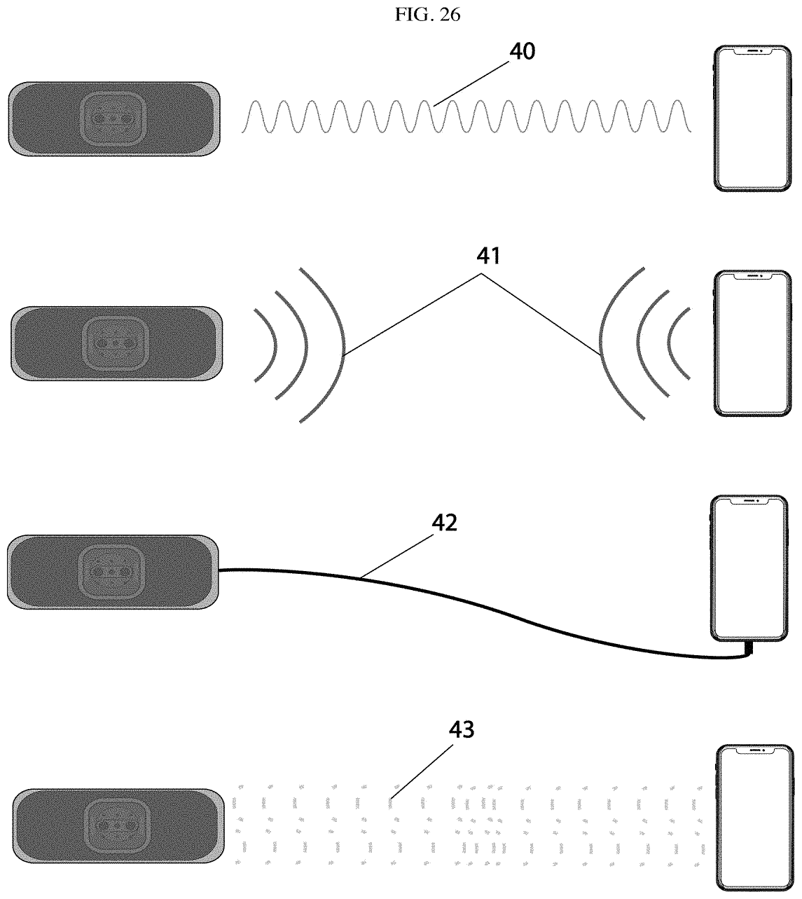

FIG. 26. In further embodiments, communication means may be any or a combination of radio-frequency communication, near-field communication, optical communication, acoustic communication, or any other communication means capable of receiving sensed signals and transmitting control signals.

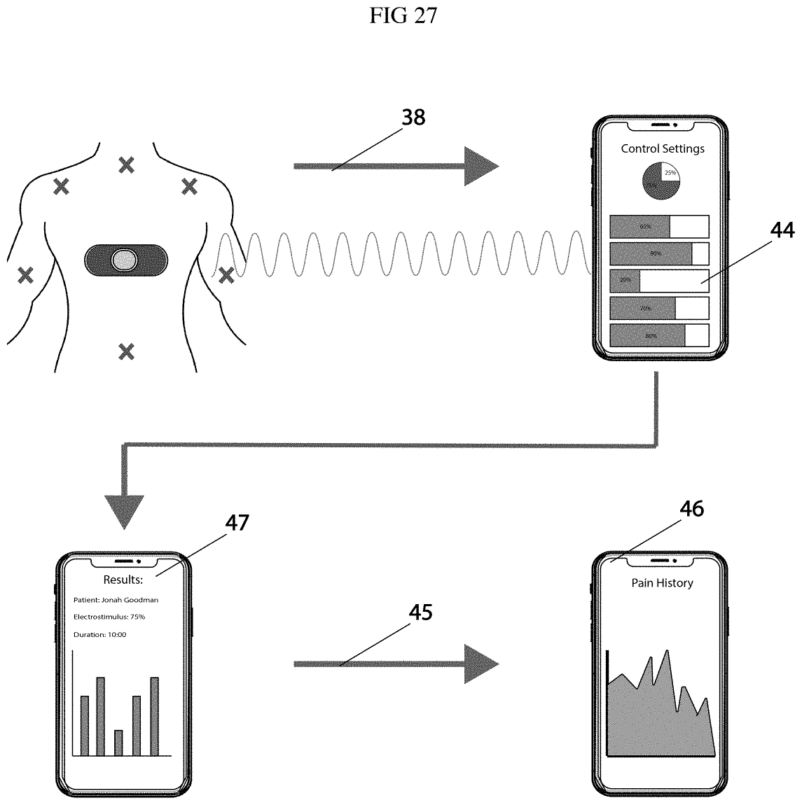

FIG. 27. In some embodiments a control subsystem comprises data communicated to an external data storage or data processing capability. In some embodiments, data communicated to an external data storage or data processing capability may include any or all of sensed signals, user inputs, control over a skin stimulator or control over a drug delivery subsystem. In further embodiments, data communicated from the control subsystem is analyzed by the external data processing capability, thus allowing the data processing capability to transmit additional information usable by the control subsystem. In additional embodiments data from multiple MMPMDs is aggregated and analyzed to generate information on the relative performance of the multiple MMPMDs. In some embodiments, a data storage or a data processing capability may be realized within a cloud service.

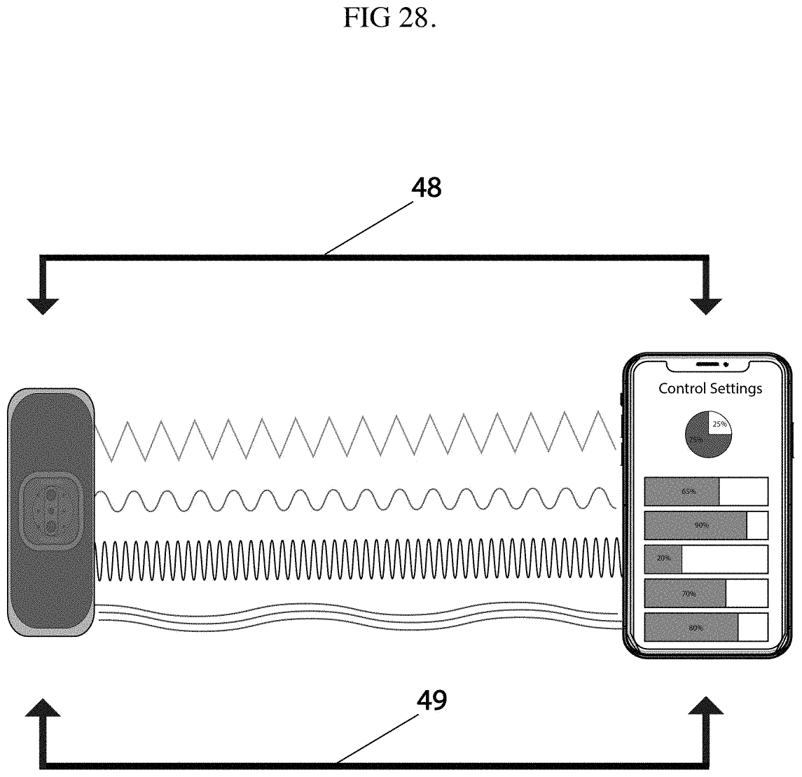

FIG. 28. In some embodiments, the control subsystem may make one or more alterations in the control of either of the skin stimulator subsystem or the drug delivery subsystem. In further embodiments, the one or more alterations are made to provide stimulus to the patient as to the relative efficacy or tolerability of the operation of the MMPMD before during or after the one or more alterations.

User Interface Subsystem

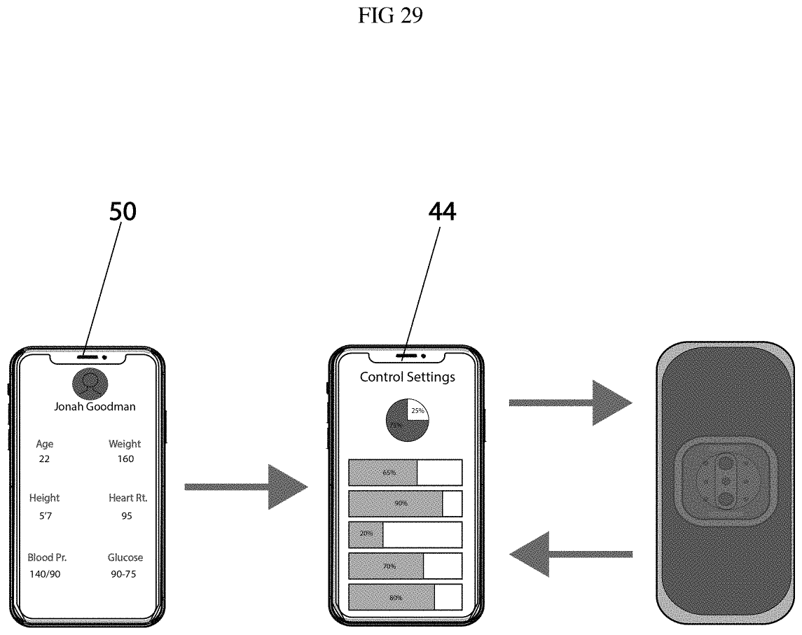

FIG. 29. A user interface subsystem may comprise any means by which a user (a patient) is provided information on the functioning of the one or more MMPMDs they are using, and by which the user may provide to the one or more MMPMDs information from user (the patient) on their preferences or their perceptions as to the functioning of the one or more MMPMDs.

In some embodiments, their perceptions may comprise perceptions of the level of pain they are experiencing. In other embodiments their perceptions may include their tolerance for the experience of using the MMPMD. In some embodiments, their preferences may comprise the time duration during which they wish to use the MMPMD. In some embodiments, the user interface subsystem may solicit input from the user in concert with one or more alterations in the control of either the skin stimulator subsystem or the drug delivery subsystem. The soliciting of input may occur at any suitable time relative to the one or more alterations in control.

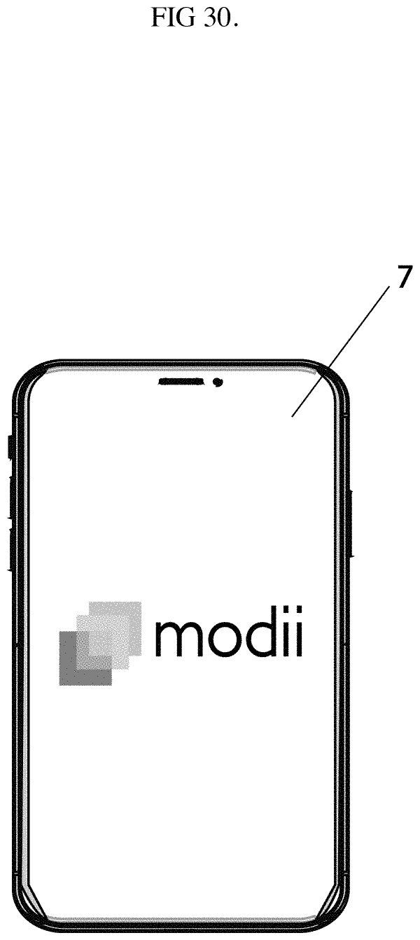

FIG. 30. In some embodiments, a user interface subsystem is implemented in part or in whole on a smart device such as a smart phone, a smart watch, or any other such smart device. In alternative embodiments, a user interface subsystem is implemented in part or in whole on a special purpose user interface device.

### Examples

### Example 1: Placement of Configuration

A person may attach the assembly in vicinity of where pain relief is desired, through the use of a releasable skin safe adhesive.

### Example 2: User Subsystem

If a person were to use the assembled configuration, then the person would be able to fully and customize and control the assembled configuration, by means of the stimulator treatment, intensity, and duration.

### Example 3: User Subsystem pt2

If a person were to use the user subsystem, then the person would be able to access their account personal account, which gives them further access to their sensor data history, the ability to order more medicated patches, communicate with a pain physician, access the community page and access white pages.

### Example 4: Medication Characteristic

If a person were to use medication A in conjunction with the assembly, then that person would take out the patch labeled medication A, remove the medicated patch, take off the release liner and attach it to the bottom of the assembly.

### Example 5: The Use of AI

If a person uses medication A, stimulation B and Intensity C, then the sensor subsystem's data output D, is stored, analyzed, and historically compared for better treatment recommendations and medication options.

### Example 6: Use of AI for Recommendation of Treatment

If a person experiences pain in area A where pain relief is desired, then a recommendation is presented, which includes stimulation B with medication C for duration D. However, if a person experiences pain in area W, then a recommendation is presented, which includes stimulation X with medication Y for duration Z.

### Example 7: Rapid Medication Depletion

If a person desires that medication A is to be depleted at a rapid pace, then a person would choose thermal stimulation B and intensity C for rapid medication depletion.

### Example 8: Inert Medication Depletion

If a person desires that medication A is to be depleted at an inert pace, then a person would not choose thermal stimulation B and intensity C for inert medication depletion.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel or may be performed at different times.

The various embodiments described above, are presented as examples only, and not as a limitation. The descriptions are not intended to limit the scope of the present technology to the forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

What is claimed is:

1. A multi-modal personalized pain management system comprising:
a sensor subsystem within a single assembly;
a skin stimulator subsystem within the single assembly;
a drug delivery subsystem within the single assembly;
a control subsystem within the single assembly; and
a user interface subsystem.

2. The multi-modal personalized pain management system of claim 1, further comprising:
the single assembly configured to be attached or worn in a vicinity where pain relief is desired.

3. The multi-modal personalized pain management system of claim 1, further comprising:
the user interface subsystem including software with instructions executed by a processor and configured to operate as part of the system or a separate computing device.

4. The multi-modal personalized pain management system of claim 1, wherein the sensor subsystem includes any of an oximeter, a pulse oximeter, a thermometer, an infrared radiation sensor, a colorimeter, a blood flow sensor, a pH sensor, a galvanic skin response sensor, an imbedded accelerometer, a biochemical sensor, an infrared radiation sensor, a colorimeter, a biochemical sensor, or a combination thereof.

5. The multi-modal personalized pain management system of claim 1, further comprising the skin stimulator subsystem including an electrical stimulation portion, a thermal stimulation portion, a mechanical stimulation portion or any combination of an electrical portion, and a thermal portion, or a combination of an electrical portion and a mechanical portion, or a combination of a thermal portion and a mechanical portion, or a combination of an electrical portion, a thermal portion, and a mechanical portion.

6. The multi-modal personalized pain management system of claim 5, wherein the electrical stimulation portion induces a current flow to a tissue.

7. The multi-modal personalized pain management system of claim 5, wherein the thermal stimulation portion induces a temperature rise or fall in a tissue.

8. The multi-modal personalized pain management system of claim 5, wherein the mechanical stimulation portion applies a force to a tissue.

9. The multi-modal personalized pain management system of claim 1, further comprising the control subsystem configured to cause a time-varying force.

10. The multi-modal personalized pain management system of claim 1, further comprising any of a reciprocating electric motor, a rotating electric motor coupled to an eccentric weight, a piston driven by an external fluid such as air or water, a piezoelectric transducer, or any other means of applying a time-varying force.

11. The multi-modal personalized pain management system of claim 1, the drug delivery subsystem comprising any of a drug dispensing mechanism, a drug dispensing control mechanism, a drug supply, a drug patch, a drug dispensing solid, a flat structure with a port and a channel, a trapped volume or any combination thereof.

12. The multi-modal personalized pain management system of claim 1, the drug delivery subsystem configured to dispense two or more types of drugs.

13. The multi-modal personalized pain management system of claim 1, the control subsystem configured to receive user inputs or sensed signals and to provide control over the skin stimulator subsystem or the drug delivery subsystem.

14. The multi-modal personalized pain management system of claim 1, further comprising the system communicating over any of an Internet network, a radio frequency, near-field communication, optical communication, acoustic communication, or any other communication means capable of receiving sensed signals and transmitting control signals, or a combination thereof.

15. The multi-modal personalized pain management system of claim 1, further comprising the user interface subsystem configured to provide information on functioning of the system and to provide information from a user on their preferences or perceptions as to the functioning of the system.

16. A method for multi-modal personalized pain management, the method comprising:
receiving sensing input by a control subsystem in an assembly;
receiving user input from a user interface by the control subsystem in the assembly; and
generating a control signal by the control subsystem to control a skin stimulator subsystem in the assembly and a drug delivery subsystem in the assembly.

17. The method for multi-modal personalized pain management of claim 16, the method further comprising:
attaching the assembly in vicinity of where pain relief is desired.

18. The method for multi-modal personalized pain management of claim 16, the method further comprising:
measuring by an infrared radiation sensor a combination of a temperature of skin and a temperature of a tissue beneath the skin.

19. The method for multi-modal personalized pain management of claim 16, the method further comprising:
measuring by a colorimeter any combination of skin color, tissue color beneath skin or color of a dye.

20. The method for multi-modal personalized pain management of claim 16, the method further comprising:
sensing by a biochemical sensor a substance exuded from skin, including any of water, sodium, potassium, oil, sebum, proteins including defensins, metabolic products including lactic acid, oxides of nitrogen, or a combination thereof.
